(19) United States
(12) Patent Application Publication
(10) Pub. No.: US 2026/0356811 A1
(43) Pub. Date: Nov. 12, 2026

(54) A PROCESS FOR PURIFICATION OF ETHOFUMESATE

(71) Applicant: ADAMA AGAN LTD., Ashdod (IL)

(72) Inventors: Omer TZOR, Kiryat-Ono (IL); Sophie DE BOTTON, Jerusalem (IL); Nir GANONYAN, Modiin (IL); Itai SHALOM, Gdera (IL); Merna ALSOUSOU, Ramla (IL)

(73) Assignee: ADAMA AGAN LTD., Ashdod (IL)

(21) Appl. No.: 19/485,577

(22) PCT Filed: May 16, 2024
(86) PCT No.: PCT/IL2024/050473

§ 371 Date: Nov. 18, 2025

Related U.S. Application Data
(60) Provisional application No. 63/467,333, filed on May 18, 2023.

(51) Int. Cl. C07D 307/82

(52) U.S. Cl. CPC C07D 307/82

(57) ABSTRACT

The present invention relates to a process for purification of crude of ethofumesate from one or more impurities selected from ethyl methanesulfonate, isobutyl methanesulfonate and any combination thereof. The process involves converting said impurities to water miscible byproducts. The invention further relates to suspension concentrate formulations comprising ethofumesate.

### FIELD OF THE INVENTION

The present invention relates to a process for purification of ethofumesate. Aspects of the present invention also relates to suspension concentrate formulations.

### BACKGROUND

Ethofumesate belongs to dihydrobenzofuran class of herbicides (chemical name: (±)-2-Ethoxy-2,3-dihydro-3,3-dimethyl-5-benzofuranol methanesulfonate). It is widely used as a pre- and post-emergence herbicide, and is commonly used in sugar beet and other beet crops as well as in turf and rye grass to control grasses and weeds. Ethofumesate acts by inhibiting the growth of meristems, retarding cell division, and limiting formation of cuticles.

While numerous methods have been reported so far for production and purification of ethofumesate, presence of methanesulfonate impurities has been a major bottleneck. Ethyl methanesulfonate (EMS) and its structurally related compound iso-butyl methanesulfonate (iBMS) are impurities, that are formed during the preparation of ethofumesate, are known to possess carcinogenic and teratogenic properties. These compounds (impurities) are known to damage DNA leading to genetic mutations, single-stranded breaks in DNA, and chromosomal aberrations. Therefore, they have been considered as mutagenic, carcinogenic, and teratogenic. For these reasons, regulations dictate that the abundancy values of these two materials should not exceed 0.1 ppm in ethofumesate.

Accordingly, need is felt of a new and improved process for purification of ethofumesate that may overcome one or more problems associated with the conventional processes. The present invention satisfies the existing needs, as well as others, and generally overcomes the deficiencies found in the state of art.

### OBJECTS

Primary object of the present invention is to provide a new and improved process that may overcome one or more limitations associated with the conventional processes for purification of ethofumesate.

It is an object of the present disclosure to provide an improved process for purification of ethofumesate that is facile, economical and industrially applicable.

Another object of the present disclosure is to provide a process for purification of ethofumesate that is safe and environment friendly.

Further aspect of the present disclosure is to provide a process for preparation of suspension concentrate formulations that is facile, economical and industrially applicable.

### SUMMARY

The present invention relates to a process for purification of ethofumesate. Aspects of the present invention also relates to suspension concentrate formulations.

The present invention disclose a process for purification of solid ethofumesate crude comprising the steps of: (a) providing a crude comprising solid ethofumesate and one or more impurities selected from ethyl methanesulfonate, isobutyl methanesulfonate and any combination thereof; (b) adding water in the presence of a base, optionally in the presence of at least one water immiscible solvent, and heating to at least 30 °C thereby converting said impurities to water miscible byproducts; and (c) discarding the aqueous phase comprising said byproducts thereby obtaining purified ethofumesate.

A plausible reaction mechanism is schematically represented hereinbelow:

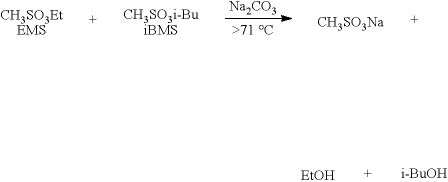

Accordingly, an aspect of the present disclosure relates to a process for purification of solid ethofumesate crude to reduce impurities.

In some embodiments, the purified ethofumesate comprises ethyl methanesulfonate in an amount less than about 0.1 ppm. In some embodiments, the purified ethofumesate comprises iso-butyl methanesulfonate in an amount less than about 0.1 ppm.

The present disclosure relates also to a process for the preparation of a suspension concentrate comprising: (a) taking at least one solid water immiscible agrochemical active ingredient having a melting point of about 30 °C to about 140 °C; (b) heating said solid agrochemical active ingredient to obtain its melted form; (c) preparing a mixture comprising water and at least one surfactant at a temperature lower than the melting point of said agrochemical active ingredient; (d) adding under shearing said melted agrochemical active ingredient to said mixture to obtain a dispersion of resolidified agrochemical active ingredient in water; and (e) optionally milling said dispersion to reduce the size of the particles.

Other aspects, advantages, and salient features of the invention will become apparent to those skilled in the art from the following detailed description, which, taken in conjunction with the exemplary embodiments of the invention.

### DETAILED DESCRIPTION

The following is a detailed description of embodiments of the present invention. The embodiments are in such detail as to clearly communicate the invention. However, the amount of detail offered is not intended to limit the anticipated variations of embodiments; on the contrary, the intention is to cover all modifications, equivalents, and alternatives falling within the spirit and scope of the present invention as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The term “byproducts” as used herein, throughout the present disclosure, denotes the hydrolysis product(s) of the impurity (ies) (Ethyl methanesulfonate (EMS), iso-butyl methanesulfonate (iBMS) and the likes) formed during the preparation of ethofumesate.

The term “solid water immiscible agrochemical active ingredient” as used herein, throughout the present disclosure, denotes agrochemically active ingredients that has solubility of less than 1000 mg/l when measured at 20 °C in water.

The term “shearing” as used herein, throughout the present disclosure refers to a mechanical force which moves the media/solution/mixture at a rapid speed creating a shear force by collisions and rolling movements. In some cases, after the addition of the melt, the shearing is maintained for at least 5 min. The amount of time sufficient for shearing after the addition of the melt is related to the rate of addition of the melt. When the rate of addition is faster, the shearing time required is longer. One skilled in the art will recognize that any one of a variety of apparatus may be used to accomplish such mixing including; for example, rotor/stator homogenizers, inline emulsifiers, static mixers, piston homogenizers, ultrasonic homogenizers, and high-speed jets or nozzles. In-line homogenizers operating at high (e.g. 24,000) revolutions per minute (“rpm”) are preferred.

In some embodiments, the numbers expressing quantities of ingredients, properties such as concentration, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

The headings and abstract of the invention provided herein are for convenience only and do not interpret the scope or meaning of the embodiments.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Accordingly, an aspect of the present disclosure relates to a process for purification of solid ethofumesate crude to reduce impurities. The process comprises the steps of: (a) providing a crude comprising solid ethofumesate and one or more impurities selected from ethyl methanesulfonate, isobutyl methanesulfonate and any combination thereof; (b) adding water in the presence of a base, optionally in the presence of at least one water immiscible solvent, and heating to at least 30 °C thereby converting said impurities to water miscible byproducts; and (c) discarding the aqueous phase comprising said byproducts thereby obtaining purified ethofumesate.

In some embodiments, the base is an organic base or an inorganic base.

In some embodiments, the organic base is selected from an amine base, a pyridine base, a piperidine base, a morpholine base, an amidine base, a guanidine base and combinations thereof. In some embodiments, the amine base is selected from a trialkylamine base, N,N,N′,N′-Tetramethylnaphthalene-1,8-diamine, and combinations thereof. In some embodiments, the trialkylamine base is selected from triethylamine, trimethylamine, tripropylamine, triisopropylamine, tributylamine, triisobutylamine, tri-2-butylamine, tri-isooctylamine di-isopropylethylamine (DIPEA), trihexylamine, 1,4-Diazabicyclo[2.2.2]octane (DABCO) and combinations thereof. In some embodiments, the pyridine base is 4-(dimethylamino)pyridine (DMAP). In some embodiments, the amidine base is selected from 1,8-Diazabicyclo[5.4.0]undec-7-ene (DBU), 1,5-Diazabicyclo[4.3.0]non-5-ene (DBN), and combinations thereof. In some embodiments, the guanidine base is selected from 1,5,7-Triazabicyclo[4.4.0]dec-5-ene (TBD), 1,1,3,3-Tetramethylguanidine (TMG), 7-Methyl-1,5,7-triazabicyclo[4.4.0]dec-5-ene (MTBD), and combinations thereof. While few exemplary organic bases have been mentioned hereinabove that can be used in the process of the present disclosure, any other organic base, as known to the skilled artisan, can also be used to serve its intended purpose.

In some embodiments, the inorganic base is selected from an alkali metal base, a hydroxy base, an alkoxy base, a carbonate base, and combinations thereof. In some embodiments, the alkali metal base is a sodium base, a lithium base, a potassium base, a magnesium base, a calcium base, and combinations thereof. In some embodiments, the potassium base is K₃PO₄, K₂HPO₄, KH₂PO₄, Na₃PO₄, Na₂HPO₄, NaH₂PO₄, and combinations thereof. In some embodiments, the hydroxy base is sodium hydroxide. In some embodiments, the carbonate base is lithium carbonate, sodium carbonate, sodium bicarbonate, potassium carbonate or potassium bicarbonate. While few exemplary inorganic bases have been mentioned hereinabove that can be used in the process of the present disclosure, any other inorganic base, as known to the skilled artisan, can also be used to serve its intended purpose.

In some embodiments, the amount of the base is from about 0.1% to about 20% by moles relative to ethofumesate crude, for example, from about 0.2% to about 15% by moles relative to ethofumesate crude, or from about 0.5% to about 12% by moles relative to ethofumesate crude, or from about 0.5% to about 10% by moles relative to ethofumesate crude, or from about 0.75% to about 5% by moles relative to ethofumesate crude. In a preferred embodiment, the amount of the base is from about 0.1% to about 5% by moles relative to ethofumesate crude.

In some embodiments, the weight ratio between ethofumesate crude and water is from about 1:0.1 to about 1:10, for example, from about 1:0.5 to about 1:7, or from about 1:0.5 to about 1:5, or from about 1:0.75 to about 1:5, or from about 1:0.75 to about 1:3. In a preferred embodiment, the weight ratio between ethofumesate and water is from about 1:0.5 to about 1:5.

The skilled artisan would appreciate that the process of the present disclosure being a solvent free process allows higher production capacity (i.e. production of more product per batch) and precludes the solvent recovery step, making the process highly economical. It should also be appreciated that while the process of the present disclosure can be done in solvent-free mode, organic solvent (a water immiscible solvent) can also be added to further improve the reaction rate.

In some embodiments, step (b) is performed in water and at least one water immiscible solvent at temperature of at least about 30 °C, for example, at a temperature ranging from about 30 °C to about 60 °C or from about 35 °C to about 55 °C. In a preferred embodiment, the step (b) is performed in water and at least one water immiscible solvent at temperature ranging from about 30 °C to about 60 °C.

In some embodiments, the weight ratio between ethofumesate crude and the water immiscible solvent is from about 1:0.1 to about 1:10, for example, from about 1:0.5 to about 1:10, or from about 1:0.5 to about 1:7, or from about 1:0.5 to about 1:5, or from about 1:0.75 to about 1:5, or from about 1:0.75 to about 1:3. In a preferred embodiment, the weight ratio between ethofumesate and the water immiscible solvent is from about 1:0.5 to about 1:5.

In some embodiments, the at least one water immiscible solvent is selected from toluene, chlorobenzene, xylene, dichloromethane, 1,2-dichloroethane, methyl-tert-butyl ether, ethyl acetate, and combinations thereof.

In some embodiments, step (b) of the process further comprises addition of a phase transfer catalyst. Presence of a phase transfer catalyst may aid in improving the rate of the reaction. While any conventional phase transfer catalyst, as known to the skilled artisan, can be used in the process of the present disclosure. In some embodiments, the phase transfer catalyst is selected from onium salts, crown ethers and polyethers.

In some embodiments, the onium salts are selected from quaternary ammonium salts, quaternary phosphonium salts, and sulfonium salts. In some embodiments, the quaternary ammonium salt is a tetrabutylammonium halide. In some embodiments, the tetrabutylammonium halide is selected from benzyltriethylammonium chloride (TEBA), tetrabutylammonium bromide (TBAB) or trioctylmethylammonium chloride (TCMAC). In some embodiments, the quaternary phosphonium salt is selected from (4-Carboxybutyl)triphenylphosphonium bromide, (Methoxymethyl)triphenylphosphonium chloride, Methyltriphenylphosphonium bromide, Methyltriphenylphosphonium iodide, Tetraphenylphosphonium bromide, Ethyltriphenylphosphonium bromide, Phosphonium(2-ethoxy-2-oxoethyl)triphenylbromide, (Carbethoxyethylidene)triphenylphosphorane, Tetrabutylphosphonium bromide, Butyltriphenylphosphonium bromide, Phosphonium(3-carboxypropyl)triphenyl bromide, Phosphonium(5-carboxypentyl)triphenyl bromide, Isopropyltriphenylphosphonium iodide, Propyltriphenylphosphonium bromide, Phosphoniumtriphenyl-2-propen-1-yl-bromide, Phosphonium(cyanomethyl)triphenyl chloride, (3-Bromopropyl)triphenylphosphonium bromide, Ethyltriphenylphosphonium iodide, Tetraphenylphosphonium chloride, Phosphonium(2-carboxyethyl)triphenyl-, bromide, Phosphonium(2-methoxy-2-oxoethyl)triphenyl bromide, Phosphonium(2-ethoxy-1-methyl-2-oxoethyl)triphenyl bromide, Phosphoniumpentyltriphenyl-, bromide, Phosphonium[(4-chlorophenyl)methyl]triphenyl chloride, Phosphonium[2-(1,3-dioxan-2-yl)ethyl]triphenyl bromide, Phosphonium(2-oxo-2-phenylethyl)triphenyl bromide, Phosphonium(bromomethyl)triphenyl, bromide, Phosphonium(chloromethyl)triphenyl chloride, Phosphonium cyclopropyltriphenyl bromide, Phosphoniumheptyltriphenyl bromide, Phosphonium(2-oxopropyl)triphenyl chloride, Phosphonium(4-ethoxy-4-oxobutyl)triphenyl bromide, (4-Nitrobenzyl)triphenylphosphonium bromide, Phosphoniumhexyltriphenyl bromide, Phosphonium(1-naphthalenylmethyl)triphenyl chloride, Phosphonium[(4-fluorophenyl)methyl]triphenyl chloride, Hexadecyltributylphosphonium bromide, Methyltriphenylphosphonium chloride, 2-Chlorobenzyltriphenylphosphonium chloride, Phosphonium, triphenyl-2-propyn-1-yl bromide, Dodecyltributylphosphonium bromide, Phosphoniumtributyl(cyanomethyl) chloride, Tetraphenylphosphonium tetraphenylborate, Benzyltriphenylphosphonium chloride, Tributylmethylphosphonium iodide, Phosphonium iodomethyl)triphenyl iodide, Phosphonium(2-oxoethyl)triphenyl chloride, Phosphonium[2-(1,1-dimethylethoxy)-2-oxoethyl]triphenyl bromide, Tetraphenylphosphonium iodide, (4-Methoxybenzyl)triphenylphosphonium chloride, Phosphoniumbutyltriphenyl chloride, Phosphonium(2-methylpropyl)triphenyl bromide, Dodecyltriphenylphosphonium bromide, Phosphonium[(3-methoxyphenyl)methyl]triphenyl chloride, Phosphonium(cyclopropylmethyl)triphenyl bromide, Phosphonium[3-(dimethylamino)propyl]triphenyl-, bromide, hydrobromide, Phosphonium, triphenyl[3-(phenylmethoxy)propyl] bromide, Phosphonium[(2,4-dichlorophenyl)methyl]triphenyl chloride, Tetradecyltriphenylphosphonium bromide, Phosphonium[(2-hydroxyphenyl)methyl]triphenyl bromide, Phosphonium[2-(1,3-dioxolan-2-yl)ethyl]triphenyl bromide, Phosphonium(2-methoxyethyl)triphenyl bromide, Phosphonium(4-bromobutyl)triphenyl bromide, Phosphonium(3-ethoxy-3-oxopropyl)triphenyl bromide, Phosphoniumtetrabutyl-tetraphenylborate, Phosphoniumtriphenyl[[2-(trimethylsilyl)ethoxy]methyl] chloride, Hydroxyethyltriphenylphosphonium chloride, Phosphonium(3-methylbutyl)triphenyl, bromide, Phosphonium[(4-methylphenyl)methyl]triphenyl chloride, Tetraethylphosphonium bromide, Tetrabutylphosphonium chloride, Phosphoniumtetrabutyl hexafluorophosphate, Phosphoniumethenyltriphenyl bromide, Phosphoniumtetraoctyl bromide, Phosphonium(methoxymethyl)triphenyl bromide, Phosphonium[(2-nitrophenyl)methyl]triphenyl bromide, Phosphonium(hydroxymethyl)triphenyl chloride, Phosphonium(2-hydroxyethyl)triphenyl bromide and combination thereof. In some embodiments, the crown ether is selected from 12-crown-4, 15-crown-5, 18-crown-6 or 21-crown-7. In some embodiments, the polyether is selected from polyethylene glycol, propylene glycol, polyethylene glycol fatty ether or polyethylene glycol alkyl phenyl ether. In some embodiments, the polyethylene glycol is polyethylene glycol-400.

In some embodiments, the amount of the phase transfer catalyst is from about 0.2% to about 10% by moles relative to ethofumesate crude, for example, from about 0.2% to about 9% by moles relative to ethofumesate crude, or from about 0.5% to about 7% by moles relative to ethofumesate crude, or from about 1% to about 7% by moles relative to ethofumesate crude. In a preferred embodiment, the amount of the phase transfer catalyst is from about 1% to about 7% by moles relative to ethofumesate crude.

In some embodiments, the step (b) of the process is performed in water at temperature ranging from about 73 °C to about 130 °C, for example, at a temperature ranging from about 73 °C to about 100 °C, or from about 75 °C to about 100 °C, or from about 80 °C to about 95 °C, or from about 85 °C to about 95 °C. In a preferred embodiment, the step (b) of the process is performed in water at temperature above 73 °C.

In some embodiments, the step (c) of discarding the aqueous phase is performed by phase separation or decantation techniques. While few exemplary techniques have been mentioned hereinabove that can be used in the discarding the aqueous phase, any other techniques, as known to the skilled artisan, can also be used to serve its intended purpose.

In some embodiments, after discarding the aqueous phase in step (c) an organic phase comprising purified ethofumesate in said solvent is obtained. While any conventional method, as known to the skilled artisan, can be used for the removal of said solvent of the present disclosure. In a preferred embodiment, said solvent is removed by distillation.

In some embodiments, the weight ratio between the aqueous phase and the organic phase is from about 1:0.1 to about 1:10, for example, from about 1:0.5 to about 1:7, or from about 1:0.5 to about 1:5, or from about 1:0.75 to about 1:5, or from about 1:0.75 to about 1:3. In a preferred embodiment, the weight ratio between the aqueous phase and the organic phase is from about 1:0.5 to about 1:5.

In some embodiments, the byproducts obtained in step (c) comprise a mesylate salt.

In some embodiments, the process comprises: (a) providing a crude comprising solid ethofumesate and one or more impurities selected from ethyl methanesulfonate, isobutyl methanesulfonate and any combination thereof; (b) adding water in the presence of about 0.1% to about 20% by moles of a carbonate base relative to ethofumesate crude, and heating to at least 73 °C thereby converting said impurities to water miscible byproducts; and (c) discarding the aqueous phase comprising said byproducts thereby obtaining purified ethofumesate.

In some embodiments, the process comprises: (a) providing a crude comprising solid ethofumesate and one or more impurities selected from ethyl methanesulfonate, isobutyl methanesulfonate and any combination thereof; (b) adding water in the presence of about 0.1% to about 20% by moles of a carbonate base relative to ethofumesate crude, in the presence of at least one water immiscible solvent and heating to at least 30 °C thereby converting said impurities to water miscible byproducts; and (c) discarding the aqueous phase comprising said byproducts thereby obtaining an organic phase comprising purified ethofumesate in said solvent.

Another aspect of the present disclosure relates to a process for the preparation of ethofumesate suspension concentrate comprising: (a) taking a purified ethofumesate as prepared in accordance with the present disclosure hereinabove; (b) optionally heating said purified ethofumesate to retain its melted form; (c) preparing a mixture comprising water and at least one surfactant at a temperature lower than the melting point of ethofumesate; (d) adding under shearing said melted ethofumesate to said mixture to obtain a dispersion of resolidified ethofumesate in water; and (e) optionally milling said dispersion to reduce size of the particles.

In some embodiments, heating in step (b) is performed at a temperature at least 3 °C higher than the melting point of ethofumesate. In some embodiments, in step (b) ethofumesate is heated to a temperature ranging from about 73 °C to about 130 °C, for example, at a temperature ranging from about 73 °C to about 100 °C, or from about 75 °C to about 100 °C, or from about 80 °C to about 95 °C, or from about 85 °C to about 95 °C. In a preferred embodiment, the step (b) of the process is performed in water at temperature from ranging about 73 °C to about 100 °C.

In some embodiments, the temperature of the mixture in step (c) is at least 10 °C lower than the melting point of ethofumesate. In some embodiments, temperature of the mixture in step (c) is from about 0 °C to about 30 °C, for example, from 2 °C to about 25 °C, or from 5 °C to about 25 °C.

In some embodiments, the dispersion of resolidified ethofumesate in step (d) has a particle size distribution with D (90) less than 1000 μm.

In some embodiments, in step (d) the rate of addition of melted ethofumesate is not more than 500 kg/hr.

Still another aspect of the present disclosure relates to a process for the preparation of a suspension concentrate comprising: (a) taking at least one solid water immiscible agrochemical active ingredient having a melting point of about 30 °C to about 140 °C; (b) heating said solid agrochemical active ingredient to obtain its melted form; (c) preparing a mixture comprising water and at least one surfactant at a temperature lower than the melting point of said agrochemical active ingredient; (d) adding under shearing said melted agrochemical active ingredient to said mixture to obtain a dispersion of resolidified agrochemical active ingredient in water; and (e) optionally milling said dispersion to reduce the size of the particles.

In some embodiments, the heating in step (b) is performed at a temperature above the melting point of said solid agrochemical active ingredient.

In some embodiments, said at least one solid water immiscible agrochemical active ingredient is selected from ethofumesate, flufenacet, cyprodinil, DNOC, dodemorph, dodemorph acetate, flusilazole, imazalil, myclobutanil, penconazole, pyraclostrobin, benfluralin, bromoxynil-heptanoate/octanoate, clodinafop-propargyl, cyhalofop-butyl,dinoterb, ethofumesate, fluoroglycofen-ethyl, flurochloridone, fluroxypyr-meptyl, haloxyfop-etotyl, ioxynil octanoate, napropamide, oxyfuorfen, pendimethalin, propanil, quizalofop-P-ethyl, propaquizafop, quizalofop-P-tefuryl, trifluralin, alpha-cypermethrin, anilofos, azinphos-ethyl, azinphos-methyl, beta-cypermethrin, chlorpyrifos, cypermethrin, alpha-endosulfan, esfenvalerate, fenoxycarb, fenvalerate, flamprop-M-isopropyl, phosmet, pyridaphenthion, resmethrin, tetramethrin, trifloxystrobin, and any combination thereof. While few exemplary solid water immiscible agrochemical active ingredient(s) have been mentioned hereinabove that can be used in the present disclosure, any other solid water immiscible agrochemical active ingredient(s), as known to the skilled artisan, can also be used to serve its intended purpose.

In some embodiments, the at least one surfactant is selected from ionic, non-ionic and amphoteric surfactants. In some embodiments, the ionic surfactant is selected from alkylarylsulfonates, phenylsulfonates, alkyl sulfates, alkylsulfonates, alkyl ether sulfates, alkylaryl ether sulfates, alkyl polyglycol ether phosphates, polyaryl phenyl ether phosphates, alkyl sulfosuccinates, olefinsulfonates, paraffinsulfonates, petroleum sulfonates, taurides, sarcosides, fatty acids, alkylnaphthalenesulfonic acids, naphthalenesulfonic acids, lignosulfonic acids, condensates of sulfonated naphthalenes with formaldehyde or with formaldehyde and phenol and, if appropriate, urea, and condensates of phenolsulfonic acid, formaldehyde and urea, lignin-sulfite waste liquor and lignosulfonates, including their alkali metal salts, alkaline earth metal salts, ammonium salts and amine salts, alkyl phosphates and polycarboxylates such as, for example, polyacrylates, maleic anhydride/olefin copolymers, and any combination thereof. In some embodiments, the non-ionic surfactant is selected from alkyl phenol alkoxylates, alcohol alkoxylates, fatty amine alkoxylates, polyoxyethylene glycerol fatty acid ester, castor oil alkoxylates, fatty acid alkoxylates, fatty acid amide alkoxylates, fatty acid polydiethanolamides, lanolin ethoxylates, fatty acid polyglycol esters, isotridecyl alcohol, fatty acid amides, methylcellulose, fatty acid esters, alkyl polyglycosides, glycerol fatty acid esters, polyethylene glycol/polypropylene glycol block copolymers, polyethylene glycol alkyl ethers, polypropylene glycol alkyl ethers, polyethylene glycol/polypropylene glycol ether block copolymers, and any combination thereof. In some embodiments, the amphoteric surfactant is selected from alkylamidopropylamine N-oxide (APAO), alkyldimethylamine N-oxide (AO), alkylbetaine (Bt), alkylamidopropylbetaine (APB), cocoamidopropyl betaine, cocoamidopropyl sultaine, lauroamphoglycinate, dihydroxyethyl tallow glycinate and any combination thereof. While few exemplary surfactant(s) have been mentioned hereinabove that can be used in the present disclosure, any other surfactant(s), as known to the skilled artisan, can also be used to serve its intended purpose.

In some embodiments, the mixture in step (c) further comprises auxiliaries. In some embodiments, the auxiliaries are selected from surfactants, dispersants, adjuvants, anti-freezing agents, anti-foaming agent, pH modifiers, co-solvents and water miscible agriculturally active ingredients. While few exemplary auxiliaries have been mentioned hereinabove that can be used in the process of the present disclosure, any other auxiliaries, as known to the skilled artisan, can also be used to serve its intended purpose.

In some embodiments, the temperature of the mixture in step (c) is at least 10 °C lower than the melting point of said agrochemical active ingredient. In some embodiments, temperature of the mixture in step (c) is from about 0 °C to about 30 °C, for example, from 2 °C to about 25 °C, or from 5 °C to about 25 °C.

In some embodiments, the mixture obtained from step (e) has a particle size distribution with D (90) less than 50 μm.

In some embodiments, the process for the preparation of the suspension concentrate after step (d) comprises a step (d1) including adding at least one other water immiscible agriculturally active ingredient to said dispersion optionally under shearing.

In some embodiments, other water immiscible agriculturally active ingredient is selected from metamitron, Phenmedipham, Desmedipham, Flufenacet, Aclonifen, Florasulam, Pendimethalin, Pyrasulfotole, Bifenox, Halauxifen-methyl, Diuron, Saflufenacil, Terbutryn, Bromoxynil, Terbuthylazine, Tembotrione, Metazachlor, Quinmerac, Pyroxasulfone, Propanil, Oxyfluorfen, Ametryn, Atrazine, Bispyribac, Cnhlorotoluron, Diflufenican, Fluometuron, Flumioxazin, Isoproturon, Linuron, Mesotrione, Propyzamide, Pyraflufen, Tebuthrion, Thidiazuron, Tralkoxydim, and any combination thereof. While few exemplary water immiscible agriculturally active ingredients have been mentioned hereinabove that can be used in the present disclosure, any other water immiscible agriculturally active ingredient(s), as known to the skilled artisan, can also be used to serve its intended purpose.

In some embodiments, the particles size of the dispersion of resolidified agrochemical active ingredient obtained in step (d) has a particle size distribution with D (90) less than 1000 μm.

In some embodiments, rate of addition of melted agrochemical active ingredient is not more than 500 kg/hr.

In some embodiments, the process for the preparation of the suspension concentrate is devoid of the step of milling and wherein the process further comprises after step (d) or step (d1) further comprises a step (d2) of adding at least one rheology modifier to said dispersion.

In some embodiments, the process for the preparation of the suspension concentrate comprises the step of milling and wherein the process further comprises, after step (e), a step (e1) of adding at least one rheology modifier to said dispersion.

In some embodiments, the process comprises: (i) heating ethofumesate to obtain its melted form; (ii) adding said melted ethofumesate under shear conditions to a mixture comprising water and at least one surfactant at a temperature lower than the melting point of ethofumesate to obtain a dispersion of resolidified ethofumesate in water; and (iii) milling said dispersion to reduce size of the particles. In some embodiments, the process comprises step (i) wherein ethofumesate is heated to a temperature of about 73 °C to about 100 °C. In some embodiments, the temperature of the mixture in step (c) is at least 10 °C lower than the melting point of ethofumesate. In some embodiments, temperature of the mixture in step (ii) is from about 0 °C to about 30 °C, for example, from 2 °C to about 25 °C, or from 5 °C to about 25 °C.

In some embodiments, the process comprises: (a) heating ethofumesate to obtain its melted form; (b) adding said melted ethofumesate under shear conditions to a mixture comprising water and at least one surfactant at a temperature lower than the melting point of ethofumesate to obtain a dispersion of resolidified ethofumesate in water; (b1) adding at least one water immiscible agriculturally active ingredient to said dispersion selected from metamitron or phenmedipham; and (c) milling said dispersion to reduce size of the particles.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the embodiments as described herein.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Example 1

1.0 gram sodium carbonate was dissolved in 200 gram water to obtain a base solution. 100 gram ethofumesate (having about 70-100 ppm EMS, and about 15-18 ppm iBMS as impurities) was added to the base solution. The resultant mixture was heated to about 100 °C, and stirred for about 8 hours while maintaining the temperature at about 100 °C. Aqueous phase from the mixture was discarded by decantation to obtain purified ethofumesate. The purified ethofumesate was further washed with water by adding 100 μm of water and heating the mixture at 100 °C for about 30 minutes followed by discarding the aqueous phase by decantation, and the resultant ethofumesate was dried at 90 °C and at 5 mbar pressure to obtain a yield of about 98% having EMS in an amount of <0.1% and iBMS in an amount of <0.1%.

Several further experiments were conducted to assess the effect of different bases, amount of base, temperature, reaction time, amount of water, and phase transfer catalyst on purity and yields of the final product (ethofumesate) following the process as mentioned in example 1 above. Process parameters and results obtained therefrom are provided in Table 1 below:

TABLE 1
Effect of process parameters on purity and yields of ethofumesate

| | Base | | Water | | | Raw material | | Final product | | | |
|---|---|---|---|---|---|---|---|---|---|---|---|
| Exp | | Amount | amount | | Temp. | EMS | iBMS | EMS | iBMS | Time | |
| No. | Base | (mol %) | (gm) | PTC | (°C) | (ppm) | (ppm) | (ppm) | (ppm) | (hrs) | Yield |
| 1 | Na₂CO₃ | 1.35 | 100 gm | — | 100 °C | 79.4 | 16.4 | <0.1 | <0.1 | 16 | 98% |
| 2 | Na₂CO₃ | 1.35 | 100 gm | — | 100 °C | 79.4 | 16.4 | <0.1 | <0.1 | 19 | 98% |
| 3 | Na₂CO₃ | 1.35 | 100 gm | — | 100 °C | 79.4 | 16.4 | <0.1 | <0.1 | 24 | 98% |
| 4 | Na₂CO₃ | 2.70 | 200 gm | — | 100 °C | 79.4 | 16.4 | <0.1 | <0.1 | 19 | 98% |
| 5 | K₂CO₃ | 1.35 | 100 gm | TBAB | 90 °C | 84.0 | 13.2 | <0.1 | 0.11 | 21 | 99% |
| | | | | 5 mol % | | | | | | | |
| 6 | K₂CO₃ | 1.35 | 100 gm | PG, | 90 °C | 79.4 | 16.4 | — | <0.1 | 21 | 98% |
| | | | | 5% w/w | | | | | | | |
| 7 | Na₂CO₃ | 1.35 | 100 gm | TBAB | 100 °C | 79.4 | 16.4 | — | <0.1 | 24 | 98% |
| | | | | 5 mol % | | | | | | | |

Based on the experiments, it could be concluded that contacting solid ethofumesate as deisclosed herein affords decomposition of methanesulfonates; the decomposition products are extracted to the aqueous phase affording a facile route for purification of ethofumesate.

It will be evident to those skilled in the art that the invention is not limited to the details of the foregoing illustrative examples and that the present invention may be embodied in other specific forms without departing from the essential attributes thereof. It is therefore desired that the present embodiments and examples be considered in all respects as illustrative and not restrictive, reference being made to the appended claims, rather than to the foregoing description and all the changes which come within the meaning and range of equivalency of the claims are therefore intended to be embraced therein.

### Example 2—Ethofumesate 500 SC

Ethofumesate tech was melted at 80 °C. Meanwhile, a homogenous, aqueous solution was prepared, containing 200 g Soprophor FL, 235 g propylene glycol, 2.5 g Silicaid AF-100 and 1800 g water. Shearing was applied to the aqueous solution, using a T50 ULTRA-TURRAX (IKA) with a S50N-G45 dispersing tool at 5000 rpm. While shearing, 2600 g of Ethofumesate melt was added to the aqueous solution at a rate of 50 g/min. Shearing was continued for 5 minutes after Ethofumesate addition to obtain a dispersion of Ethofumesate, with a particle size distribution with D (90)<300 μm. The obtained dispersion was then milled with a MultiLab DYNO mill (WAB), using 1.0-1.2 mm zirconia grinding beads, to a final size distribution of D (90)<7 μm. To complete the formulation, 25 g Silicaid AF-100 and 2.7% Xanthan-gum solution were added to a final viscosity of 1200-1800 cP (approximately 380 g). The formulation was topped with water to reach a final concentration of 500 g/L of Ethofumesate.

### Example 3—Ethofumesate 150 Metamitron 350 SC

Ethofumesate tech was melted at 80 °C. Meanwhile, a homogenous, aqueous solution was prepared, containing 172 g Soprophor FL, 172 g Ethylan NS 500 K, 250 g propylene glycol, 5 g Silicaid AF-100 and 1780 g water. Shearing was applied to the aqueous solution, using a T50 ULTRA-TURRAX (IKA) with a S50N-G45 dispersing tool at 5000 rpm. While shearing, 780 g of Ethofumesate melt was added to the aqueous solution at a rate of 50 g/min. Shearing was continued for 5 minutes after Ethofumesate addition to obtain a dispersion of Ethofumesate, with a particle size distribution with D (90)<500 μm. 1790 g of Metamitron tech powder was added while shearing. The obtained dispersion mixture was then milled with a MultiLab DYNO mill (WAB), using 1.0-1.2 mm zirconia grinding beads, to a final size distribution of D (90)<6 μm. To complete the formulation, 5 g Silicaid AF-100 and 2.7% Xanthan-gum solution were added to a final viscosity of 1200-1800 cP (approximately 520 g). The formulation was topped with water to reach a final concentration of 500 g/L of Ethofumesate.

### Advantages

The present disclosure provides a new and improved process that may overcome one or more limitations associated with the conventional processes for purification of ethofumesate.

The present disclosure provides an improved process for purification of ethofumesate that is facile, economical and industrially applicable.

The present disclosure provides a process for purification of ethofumesate that is safe and environment friendly.

The present disclosure provides a process for preparation of a suspension concentrate formulation that is facile, economical and industrially applicable.

1. A process for purification of solid ethofumesate crude comprising the steps of:
(a) providing a crude comprising solid ethofumesate and one or more impurities selected from ethyl methanesulfonate, isobutyl methanesulfonate and any combination thereof;
(b) adding water in the presence of a base, optionally in the presence of at least one water immiscible solvent, and heating to at least 30 °C thereby converting said impurities to water miscible byproducts; and
(c) discarding the aqueous phase comprising said byproducts thereby obtaining purified ethofumesate.

2. The process according to claim 1, wherein the at least one water immiscible solvent is selected from toluene, chlorobenzene, xylene, dichloromethane, 1,2-dichloroethane, methyl-tert-butyl ether, ethyl acetate and combinations thereof.

3. The process according to claim 1 or 2, wherein said byproducts comprise a mesylate salt.

4. The process according to any one of claims 1-3, wherein the base is an organic base or an inorganic base.

5. The process according to claim 4, wherein the organic base is selected from an amine base, a pyridine base, a piperidine base, a morpholine base, an amidine base, a guanidine base and combinations thereof.

6. The process according to claim 5, wherein the amine base is selected from a trialkylamine base, N,N,N′,N′-Tetramethylnaphthalene-1,8-diamine, and combinations thereof.

7. The process according to claim 6, wherein the trialkylamine base is selected from triethylamine, trimethylamine, tripropylamine, triisopropylamine, tributylamine, triisobutylamine, tri-2-butylamine, tri-isooctylamine di-isopropylethylamine (DIPEA), trihexylamine, 1,4-Diazabicyclo[2.2.2]octane (DABCO) and combinations thereof.

8. The process according to claim 5, wherein the pyridine base is 4-(dimethylamino)pyridine (DMAP).

9. The process according to claim 5, wherein the amidine base is selected from 1,8-Diazabicyclo[5.4.0]undec-7-ene (DBU), 1,5-Diazabicyclo[4.3.0]non-5-ene (DBN), and combinations thereof.

10. The process according to claim 5, wherein the guanidine base is selected from 1,5,7-Triazabicyclo[4.4.0]dec-5-ene (TBD), 1,1,3,3-Tetramethylguanidine (TMG), 7-Methyl-1,5,7-triazabicyclo[4.4.0]dec-5-ene (MTBD), and combinations thereof.

11. The process according to claim 4, wherein the inorganic base is selected from an alkali metal base, a hydroxy base, an alkoxy base, a carbonate base, and combinations thereof.

12. The process according to claim 11, wherein the alkali metal base is a sodium base, a lithium base, a potassium base, a magnesium base, a calcium base, and combinations thereof.

13. The process according to claim 12, wherein the potassium base is K₃HPO₄, K₂HPO₄, KH₂PO₄, Na₃PO₄, Na₂HPO₄, NaH₂PO₄, and combinations thereof.

14. The process according to claim 11, wherein the hydroxy base is sodium hydroxide.

15. The process according to claim 11, wherein the carbonate base is lithium carbonate, sodium carbonate, sodium bicarbonate, potassium carbonate or potassium bicarbonate.

16. The process according to any one of claims 1-15, wherein the amount of the base is from about 0.1% to about 20% relative to ethofumesate crude, by moles.

17. The process according to any one of claims 1-16, wherein step (b) of the process further comprises addition of a phase transfer catalyst.

18. The process according to claim 17, wherein the phase transfer catalyst is selected from onium salts, crown ethers and polyethers.

19. The process according to claim 18, wherein the onium salts are selected from quaternary ammonium salts, quaternary phosphonium salts, and sulfonium salts.

20. The process according to claim 19, wherein the quaternary ammonium salt is a tetrabutylammonium halide.

21. The process according to claim 20, wherein the tetrabutylammonium halide is selected from benzyltriethylammonium chloride (TEBA), tetrabutylammonium bromide (TBAB) or trioctylmethylammonium chloride (TCMAC).

22. The process according to claim 19, wherein the quaternary phosphonium salt is selected from (4-Carboxybutyl)triphenylphosphonium bromide, (Methoxymethyl) triphenylphosphonium chloride, Methyltriphenylphosphonium bromide, Methyltriphenyl phosphonium iodide, Tetraphenylphosphonium bromide, Ethyltriphenylphosphonium bromide, Phosphonium(2-ethoxy-2-oxoethyl)triphenylbromide, (Carbethoxyethylidene) triphenylphosphorane, Tetrabutylphosphonium bromide, Butyltriphenylphosphonium bromide, Phosphonium(3-carboxypropyl)triphenyl bromide, Phosphonium(5-carboxypentyl)triphenyl bromide, Isopropyltriphenylphosphonium iodide, Propyltriphenylphosphonium bromide, Phosphoniumtriphenyl-2-propen-1-yl-bromide, Phosphonium(cyanomethyl)triphenyl chloride, (3-Bromopropyl)triphenylphosphonium bromide, Ethyltriphenylphosphonium iodide, Tetraphenylphosphonium chloride, Phosphonium(2-carboxyethyl)triphenyl-, bromide, Phosphonium(2-methoxy-2-oxoethyl)triphenyl bromide, Phosphonium(2-ethoxy-1-methyl-2-oxoethyl)triphenyl bromide, Phosphoniumpentyltriphenyl-, bromide, Phosphonium[(4-chlorophenyl)methyl]triphenyl chloride, Phosphonium[2-(1,3-dioxan-2-yl)ethyl]triphenyl bromide, Phosphonium(2-oxo-2-phenylethyl)triphenyl bromide, Phosphonium(bromomethyl)triphenyl, bromide, Phosphonium(chloromethyl)triphenyl chloride, Phosphonium cyclopropyltriphenyl bromide, Phosphoniumheptyltriphenyl bromide, Phosphonium(2-oxopropyl)triphenyl chloride, Phosphonium(4-ethoxy-4-oxobutyl)triphenyl bromide, (4-Nitrobenzyl)triphenylphosphonium bromide, Phosphoniumhexyltriphenyl bromide, Phosphonium(1-naphthalenylmethyl)triphenyl chloride, Phosphonium[(4-fluorophenyl)methyl]triphenyl chloride, Hexadecyltributylphosphonium bromide, Methyltriphenylphosphonium chloride, 2-Chlorobenzyltriphenylphosphonium chloride, Phosphonium, triphenyl-2-propyn-1-yl bromide, Dodecyltributylphosphonium bromide, Phosphoniumtributyl(cyanomethyl) chloride, Tetraphenylphosphonium tetraphenylborate, Benzyltriphenylphosphonium chloride, Tributylmethylphosphonium iodide, Phosphonium iodomethyl)triphenyl iodide, Phosphonium(2-oxoethyl)triphenyl chloride, Phosphonium[2-(1,1-dimethylethoxy)-2-oxoethyl]triphenyl bromide, Tetraphenylphosphonium iodide, (4-Methoxybenzyl)triphenylphosphonium chloride, Phosphoniumbutyltriphenyl chloride, Phosphonium(2-methylpropyl)triphenyl bromide, Dodecyltriphenylphosphonium bromide, Phosphonium[(3-methoxyphenyl)methyl]triphenyl chloride, Phosphonium(cyclopropylmethyl)triphenyl bromide, Phosphonium[3-(dimethylamino)propyl]triphenyl-, bromide, hydrobromide, Phosphonium, triphenyl[3-(phenylmethoxy)propyl] bromide, Phosphonium[(2,4-dichlorophenyl)methyl]triphenyl chloride, Tetradecyltriphenylphosphonium bromide, Phosphonium[(2-hydroxyphenyl)methyl]triphenyl bromide, Phosphonium[2-(1,3-dioxolan-2-yl)ethyl]triphenyl bromide, Phosphonium(2-methoxyethyl)triphenyl bromide, Phosphonium(4-bromobutyl)triphenyl bromide, Phosphonium(3-ethoxy-3-oxopropyl)triphenyl bromide, Phosphoniumtetrabutyl-tetraphenylborate, Phosphoniumtriphenyl[[2-(trimethylsilyl)ethoxy]methyl] chloride, Hydroxyethyltriphenylphosphonium chloride, Phosphonium(3-methylbutyl)triphenyl, bromide, Phosphonium[(4-methylphenyl)methyl]triphenyl chloride, Tetraethylphosphonium bromide, Tetrabutylphosphonium chloride, Phosphoniumtetrabutyl hexafluorophosphate, Phosphoniumtetraoctyl bromide, Phosphoniumethenyltriphenyl bromide, Phosphonium(methoxymethyl)triphenyl bromide, Phosphonium[(2-nitrophenyl)methyl]triphenyl bromide, Phosphonium(hydroxymethyl)triphenyl chloride, or Phosphonium, (2-hydroxyethyl)triphenyl bromide.

23. The process according to claim 18, wherein the crown ether is selected from 12-crown-4, 15-crown-5, 18-crown-6 or 21-crown-7.

24. The process according to claim 18, wherein the polyether is selected from polyethylene glycol, propylene glycol, polyethylene glycol fatty ether or polyethylene glycol alkyl phenyl ether.

25. The process according to claim 24, wherein the polyethylene glycol is polyethylene glycol-400.

26. The process according to any one of claims 17-25, wherein the amount of the phase transfer catalyst is from about 0.2% to about 10% relative to ethofumesate crude, by moles.

27. The process according to any one of claims 1-26, wherein step (b) of the process is performed in water at temperature above 73 °C.

28. The process according to any one of claims 1-27, wherein step (b) of the process is performed in water at temperature of about 73 °C to about 100 °C.

29. The process according to any one of claims 1-28, wherein step (b) of the process is performed in water at temperature form about 75 °C to about 100 °C.

30. The process according to any one of claims 1-29, wherein discarding the aqueous phase in step (c) is performed by phase separation or decantation.

31. The process according to any one of claims 1-30, wherein the weight ratio between ethofumesate crude and the water is from about 1:0.1 to about 1:10.

32. The process according to claim 31, wherein the weight ratio between ethofumesate crude and the water is from about 1:0.5 to about 1:5.

33. The process according to any one of claims 1-26, wherein step (b) is performed in water and at least one water immiscible solvent at temperature of at least about 30 °C.

34. The process according to claim 33, wherein the temperature is of about 30 °C to about 60 °C.

35. The process according to claim 33 or 34, wherein after discarding the aqueous phase in step (c) organic phase comprising purified ethofumesate in said solvent is obtained.

36. The process according to claim 35, wherein said solvent is removed by distillation.

37. The process according to any one of claims 33-36, wherein the weight ratio between ethofumesate crude and the water immiscible solvent is from about 1:0.5 to about 1:10.

38. The process according to claim 37, wherein the weight ratio between ethofumesate crude and the water immiscible solvent is from about 1:0.5 to about 1:5.

39. The process according to any one of claims 33-38, wherein the weight ratio between the aqueous phase and the organic phase is from about 1:0.1 to about 1:10.

40. The process according to any one of claim 39, wherein the weight ratio between the aqueous phase and the organic phase is from about 1:0.5 to about 1:5.

41. The process according to any one of claims 1-32, wherein the process comprises:
(a) providing a crude comprising solid ethofumesate and one or more impurities selected from ethyl methanesulfonate, isobutyl methanesulfonate and any combination thereof;
(b) adding water in the presence of about 0.1% to about 20% of a carbonate base by moles relative to ethofumesate crude, and heating to at least 73 °C thereby converting said impurities to water miscible byproducts; and
(c) discarding the aqueous phase comprising said byproducts thereby obtaining purified ethofumesate.

42. The process according to any one of claims 33-40, wherein the process comprises:
(a) providing a crude comprising solid ethofumesate and one or more impurities selected from ethyl methanesulfonate, isobutyl methanesulfonate and any combination thereof;
(b) adding water in the presence of about 0.1% to about 20% of a carbonate base by moles relative to ethofumesate crude, in the presence of at least one water immiscible solvent and heating to at least 30 °C thereby converting said impurities to water miscible byproducts; and
(c) discarding the aqueous phase comprising said byproducts thereby obtaining an organic phase comprising purified ethofumesate in said solvent.

43. A process for the preparation of ethofumesate suspension concentrate comprising:
(a) obtaining purified ethofumesate according to any one of claims 1-42;
(b) optionally heating said purified ethofumesate to retain its melted form;
(c) preparing a mixture comprising water and at least one surfactant at a temperature lower than the melting point of ethofumesate;
(d) adding under shearing said melted ethofumesate to said mixture to obtain a dispersion of resolidified ethofumesate in water; and
(e) optionally milling said dispersion to reduce the size of the particles.

44. The process according to claim 43, wherein the heating in step (b) is performed at a temperature at least 3 °C higher than the melting point of ethofumesate.

45. The process according to claim 43 or 44, wherein in step (b) ethofumesate is heated to a temperature of about 73 °C to about 100 °C.

46. The process according to any one of claims 43-45, wherein the temperature of the mixture in step (c) is at least 10 °C lower than the melting point of ethofumesate.

47. The process according to claim 46, wherein the temperature of said mixture in step (c) is of about 0 °C to about 30 °C.

48. The process according to any one of claims 43-47, wherein the rate of addition of said melted ethofumesate is not more than 500 kg/hr.

49. The process according to any one of claims 43-48, wherein the dispersion of resolidified ethofumesate in step (d) has a particle size distribution with D (90) less than 1000 μm.

50. A process for the preparation of a suspension concentrate comprising:
(a) taking at least one solid water immiscible agrochemical active ingredient having a melting point of about 30 °C to about 140 °C;
(b) heating said solid agrochemical active ingredient to obtain its melted form;
(c) preparing a mixture comprising water and at least one surfactant at a temperature lower than the melting point of said agrochemical active ingredient;
(d) adding under shearing said melted agrochemical active ingredient to said mixture to obtain a dispersion of resolidified agrochemical active ingredient in water; and
(e) optionally milling said dispersion to reduce the size of the particles.

51. The process according to claim 50, wherein the heating in step (b) is performed at a temperature above the melting point of said solid agrochemical active ingredient.

52. The process according to claim 50 or 51, wherein said at least one solid water immiscible agrochemical active ingredient is selected from ethofumesate, flufenacet, cyprodinil, DNOC, dodemorph, dodemorph acetate, flusilazole, imazalil, myclobutanil, penconazole, pyraclostrobin, benfluralin, bromoxynil-heptanoate/octanoate, clodinafop-propargyl, cyhalofop-butyl, dinoterb, ethofumesate, fluoroglycofen-ethyl, flurochloridone, fluroxypyr-meptyl, haloxyfop-etotyl, ioxynil octanoate, napropamide, oxyfuorfen, pendimethalin, propanil, quizalofop-P-ethyl, propaquizafop, quizalofop-P-tefuryl, trifluralin, alpha-cypermethrin, anilofos, azinphos-ethyl, azinphos-methyl, beta-cypermethrin, chlorpyrifos, cypermethrin, alpha-endosulfan, esfenvalerate, fenoxycarb, fenvalerate, flamprop-M-isopropyl, phosmet, pyridaphenthion, resmethrin, tetramethrin, trifloxystrobin, and any combination thereof.

53. The process according to any one of claims 43-52, further comprising after step (d) step (d1) adding at least one other water immiscible agriculturally active ingredient to said dispersion, optionally, under shearing.

54. The process according to claim 53, wherein said other water immiscible agriculturally active ingredient is selected from metamitron, Phenmedipham, Desmedipham, Flufenacet, Aclonifen, Florasulam, Pendimethalin, Pyrasulfotole, Bifenox, Halauxifen-methyl, Diuron, Saflufenacil, Terbutryn, Bromoxynil, Terbuthylazine, Tembotrione, Metazachlor, Quinmerac, Pyroxasulfone, Propanil, Oxyfluorfen, Ametryn, Atrazine, Bispyribac, Chlorotoluron, Diflufenican, Fluometuron, Flumioxazin, Isoproturon, Linuron, Mesotrione, Propyzamide, Pyraflufen, Tebuthrion, Thidiazuron, Tralkoxydim.

55. The process according to any one of claims 43-54, wherein the at least one surfactant is selected from ionic, non-ionic and amphoteric surfactants.

56. The process according to any one of claims 43-55, wherein said mixture in step (c) is further comprising auxiliaries.

57. The process according to claim 56, wherein the auxiliaries are selected from surfactants, dispersants, adjuvants, anti-freezing agent, anti-foaming agent, pH modifiers, co-solvents and water miscible agriculturally active ingredients.

58. The process according to any one of claims 50-57, wherein the temperature of the mixture in step (c) is at least 10 °C lower than the melting point of said agrochemical active ingredient.

59. The process according to claim 58, wherein the temperature of said mixture in step (c) is of about 0 °C to about 30 °C.

60. The process according to any one of claims 50-59, wherein the rate of addition of said melted agrochemical active ingredient is not more than 500 kg/hr.

61. The process according to any one of claims 50-60, wherein the particles size of the dispersion of resolidified agrochemical active ingredient obtained in step (d) has a particle size distribution with D (90) less than 1000 μm.

62. The process according to any one of claims 43-61, wherein said mixture obtained from step (e) has a particle size distribution with D (90) less than 50 μm.

63. The process according to any one of claims 43-62, wherein the process is devoid of the step of milling and wherein the process further comprises, after step (d) or step (d1), a step (d2) of adding at least one rheology modifier to said dispersion.

64. The process according to any one of claims 43-62, wherein the process comprises the step of milling and wherein the process further comprises, after step (e), a step (e1) of adding at least one rheology modifier to said dispersion.

65. The process according to any one of claims 50-64, wherein the process comprises:
(i) heating ethofumesate to obtain its melted form;
(ii) adding said melted ethofumesate under shear conditions to a mixture comprising water and at least one surfactant at a temperature lower than the melting point of ethofumesate to obtain a dispersion of resolidified ethofumesate in water; and
(iii) milling said dispersion to reduce the size of the particles.

66. The process according to claim 65, wherein in step (i) ethofumesate is heated to a temperature of about 73 °C to about 100 °C.

67. The process according to claim 50 or 65, wherein the temperature of said mixture in step (ii) is of about 0 °C to about 30 °C.

68. The process according to any one of claims 50-67, wherein the process comprises:
(a) heating ethofumesate to obtain its melted form;
(b) adding said melted ethofumesate under shear conditions to a mixture comprising water and at least one surfactant at a temperature lower than the melting point of ethofumesate to obtain a dispersion of resolidified ethofumesate in water;
(b1) adding at least one water immiscible agriculturally active ingredient to said dispersion selected from metamitron or phenmedipham; and
(c) milling said dispersion to reduce the size of the particles.

69. The suspension concentrate produced by the process according to any one of claims 43-68.
